(19) United States
(12) Patent Application Publication
(10) Pub. No.: US 2018/0085082 A1
(43) Pub. Date: Mar. 29, 2018

(54) X-RAY IMAGING APPARATUS

(71) Applicant: SHIMADZU CORPORATION, Kyoto-Shi (JP)

(72) Inventor: Masahiro KAWANO, Kyoto-Shi (JP)

(73) Assignee: SHIMADZU CORPORATION, Kyoto-Shi (JP)

(21) Appl. No.: 15/273,833

(22) Filed: Sep. 23, 2016

(51) Int. Cl. A61B 6/00; A61B 6/06

(52) U.S. Cl. CPC A61B 6/542; A61B 6/06

(57) ABSTRACT

An X-ray imaging apparatus comprises a distance detector 23, a distance calculation element 37 and a visible light source control element 33. The distance calculation element 37 calculates the distance from the visible light lamp 19 to the body surface of the subject based on the distance measured by the distance detector 23. The visible light source control element 33 controls a light intensity of a visible light emitted from the visible light source based on the calculated distance by the distance calculation element 37. The operator visually recognizes a brightness of the exposure field of the visual light, and can easily confirm that the visible light lamp has shifted to a suitable position for the X-ray imaging.

### CROSS REFERENCE TO RELATED APPLICATIONS

This application relates to, but does not claim priority from, JP Ser. No. JP2014-074108 filed Mar.31, 2014, the entire contents of which are incorporated by reference.

### FIGURE SELECTED FOR PUBLICATION

FIG. 3.

### BACKGROUND OF THE INVENTION

### Field of the Invention

The present invention relates to an X-ray imaging apparatus that acquires an X-ray image of a subject by irradiating X-ray and particularly relates to the X-ray imaging apparatus having a collimator that limits the X-ray beam.

### Description of the Related Art

In medical sites, the X-ray imaging apparatus that images the subject's X-ray image by irradiating X-ray to the subject is being applied. Referring to FIG. 9, a conventional X-ray imaging apparatus 101 comprises a table 103, an X-ray tube 105, an X-ray detector 107, a collimator 109, an image generation element 111 and a monitor 113.

The subject M lies on the table 103 in a horizontal posture. The X-ray tube 105 irradiates X-rays to the subject M. The X-ray tube 105 and the X-ray detector 107 are in-place facing each other sandwiching the table 103. The X-ray detector 107 detects the X-ray, irradiated to the subject M from the X-ray tube 105, that transmits through the subject M and converts to an electric signal therefrom and then outputs as a detection signal. A flat panel display (FPD) is used as an example of the X-ray detector. The image generation element 111 is installed in the latter part of the X-ray detector 107. The image generation element 111 acquires an X-ray image of the subject M based on the X-ray detection signal output from the X-ray detector 107. The acquired X-ray image is displayed on the monitor 113.

The collimator 109 installed below the X-ray tube 105 limits X-ray beam being irradiated from the X-ray tube 105 to e.g., a pyramid-like cone shape. The collimator 109 has two pairs of shielding boards shiftable mirror-image-symmetrically at which the center axis of the X-ray beam is the baseline thereof. Broadening of the X-ray beam irradiated from the X-ray tube 105 can be adjusted by shifting the shielding boards. An exposed amount of X-rays to the subject M can be suppressed with irradiating X-rays only to the associated region of the subject M by adjusting broadening of the X-ray beam.

In addition, a visible light source is installed to the collimator 109. As an example of visible light source, such as e.g. a halogen lamp can be applied to emit the visible light to the subject M. Then, the X-ray imaging apparatus 101 can structurally change the distance between the visible light and the subject M corresponding to the X-ray imaging condition, e.g., the region of the subject M to which the X-ray is irradiated.

The visible light emitted from the visible light source is also limited by the shielding boards of the collimator 109 as well as the X-rays irradiated from the X-ray tube 105. Then, the limited visible light beam can illuminate only a part of the subject M. Specifically, the part of the subject illuminated with the visible light coincides with the region of the subject M targeted by the X-ray beam being irradiated from the X-ray tube 105. With having the visible light, the operator can predict the region of the subject M to be irradiated by the X-ray beam prior to the irradiation of the X-ray (refer to e. g., Patent Document 1, Patent Document 2.)

The operator irradiates the visible light beam from the visible light source to the subject M prior to an X-ray imaging. Then, the operator adjusts the position and the region (exposure field) to be irradiated with the visible light by opening-and-closing the shielding boards of the collimator 109 and shifting the X-ray tube 105 while keeping the irradiation of the visible light. After adjusted the exposure field, the X-ray can be irradiated to the desired region of the subject M by irradiating the X-ray from the X-ray tube 105.

### PRIOR ART RELATED ART DOCUMENTS

### Patent Document

Patent Document 1: JP Patent Published 1-111-335267 A

Patent Document 2: JP Patent Published 2011-104154

### ASPECTS AND SUMMARY OF THE INVENTION

### Objects to be Solved

Nevertheless, in the case of a conventional example having such structure, following problems are remained to be solved.

Specifically, according to the conventional X-ray imaging apparatus, when the distance which is from the visual light source to the body surface of the subject is long, a brightness of the visual light beam being irradiated from the visible light source to the subject decreases, so that the visual recognition of the exposure field may be difficult.

In such case, the brightness of the exposure field illuminated with the visible light beam can be intensified by increasing the light intensity of the visible light irradiated from the visible light source corresponding to the distance from the visible light source to the body surface of the subject. However, when the distance from the visible light source to the body surface of the subject becomes far, the light intensity of the visible light may be too intensified. Should the light intensity of the visible light is too intensified; a life span of components, e.g., a halogen lamp, constituting the visible light source can be shortened. In addition, in accordance with increase of the light intensity of the visible light, temperature of the outer housing of e.g., the collimator, the X-ray imaging apparatus and so forth should rise, so that it is concerned that the operator may get a burn on touching the outer housing or the outer housing of the X-ray imaging apparatus may deteriorate.

Further, according to the conventional X-ray imaging apparatus, the distance from the visible light source to the body surface must be measured using manually e.g., a tape measure. Accordingly, the measurement of the distance from the visible light source to the body surface is laborious. In addition, since such measurement uses the tape measure, the measured distance may not meet the satisfactory accuracy. Accordingly, the exposure field of the visible light and the exposure field of the X-ray do not coincide well, so that it is difficult to obtain the X-ray image in which the associated region of the subject may not suitably imaged.

Considering such circumstances, the purpose of the present invention is to provide an X-ray imaging apparatus capable of executing an X-ray imaging efficiently with cutting burden to the operator who measures the distance from the visible light source to the subject.

### Means for Solving the Problem

The present invention constitutes the following structure to achieve such purpose.

Specifically, an X-ray imaging apparatus of the present invention comprises: an X-ray source that irradiate the X-ray to a subject; a detection means that detects the X-ray irradiated from the X-ray source and transmitting through the subject; an image generation means that generates an X-ray image based on the detection signal output from the X-ray detection means; a shielding board mounted to a collimator installed between the X-ray source and the X-ray detection means adjusts an X-ray exposure field irradiated from the X-ray source; a visible light source mounted to the collimator emits a visible light to the subject for recognizing visually the exposure field; a measurement means that measure the distance from the visible light source to the body surface of the subject; and a visible light source control means that controls blinking of the visible light corresponding to a difference between the distance, from the visible light source to the body surface of the subject, measured by the distance measurement means and a predetermined target value.

### Action and Effect

The X-ray imaging apparatus according to the present invention comprises the distance measurement means and the visible light source control means. The measurement means measures the distance from the visible light source to the body surface of the subject. Specifically, the operator does not need to measure manually the distance from the visible light source to the body surface of the subject. Consequently, labor and burden needed to the X-ray imaging decrease, so that an efficient diagnosis can be performed.

And, the visible light source control means that controls blinking of the visible light source based on the difference between the distance, from the visible light source to the body surface of the subject, measured by the distance measurement means and a predetermined target value. Accordingly, the operator visually recognizes the exposure field of the visual light, so that the operator can confirm that the visible light source has shifted to the suitable position and the visually recognized exposure field is the optimal exposure field for the X-ray imaging. Specifically, the operator does not have to shift the line of sight to confirm the optimal position of the exposure field for the X-ray imaging. Accordingly, labor and burden to the operator to confirm the optimal position of the exposure field for the X-ray imaging decrease so that the suitable X-ray image for diagnosis can be easily and efficiently obtained.

And, according to the aspect of the above described invention, it is preferable that the visible light source control means that controls the light intensity of the visible light, which the visible light source emits, corresponding to the difference between the distance, from the visible light source to the body surface of the subject, measured by the distance measurement means and a predetermined target value.

### Action and Effect

According to the X-ray imaging apparatus of the present invention, the visible light source control means that controls the light intensity of the visible light, which the visible light source emits, corresponding to the difference between the distance, from the visible light source to the body surface of the subject, measured by the distance measurement means and a predetermined target value. Accordingly, the operator can easily confirm, without shifting the line of sight from the exposure field, that the visible light source has shifted to the suitable position and the visually recognized exposure field is the optimal exposure field for the X-ray imaging. Accordingly, labor and burden to the operator to confirm the optimal position of the exposure field for the X-ray imaging decrease so that the suitable X-ray image for diagnosis can be easily and efficiently obtained.

In addition, according to the aspect of the above described invention, it is preferable that the visible light source control means that controls the blinking aspect of the visible light, which the visible light source emits, corresponding to the difference between the distance, from the visible light source to the body surface of the subject, measured by the distance measurement means and a predetermined target value thereof.

### Action and Effect

According to the X-ray imaging apparatus of the present invention, the visible light source control means that controls the blinking aspect of the visible light, which the visible light source emits, corresponding to the difference between the distance, from the visible light source to the body surface of the subject, measured by the distance measurement means and a predetermined target value thereof Accordingly, the operator can easily confirm, without shifting own visual sight from the exposure field and by referring to the blinking aspect of the visible illuminating the exposure field, that the visually recognized exposure field is the optimal exposure field for the X-ray imaging. Accordingly, labor and burden to the operator to confirm the optimal position of the exposure field for the X-ray imaging decrease so that the suitable X-ray image for diagnosis can be easily and efficiently obtained.

In addition, according to the aspect of the above described invention, it is preferable that the visible light source control means that controls the number of blinking of the visible light per time unit, which the visible light source emits, corresponding to the difference between the distance, from the visible light source to the body surface of the subject, measured by the distance measurement means and a predetermined target value.

### Action and Effect

According to the X-ray imaging apparatus of the present invention, the visible light source control means that controls the number of blinking of the visible light per time unit, which the visible light source emits, corresponding to the difference between the distance, from the visible light source to the body surface of the subject, measured by the distance measurement means and a predetermined target value thereof. Accordingly, the operator can easily confirm, without shifting own visual sight from the exposure field and by referring to the number of blinking of the visible light per time unit illuminating the exposure field, that the visually recognized exposure field is the optimal exposure field for the X-ray imaging. Accordingly, labor and burden to the operator to confirm the optimal position of the exposure field for the X-ray imaging decrease so that the suitable X-ray image for diagnosis can be more easily and efficiently obtained.

In addition, according to the aspect of the above described invention, the distance measurement means includes a distance detector and a distance calculation means, the distance detector detects the distance from own position at which the distance detector is mounted to the collimator to the body surface of the subject, the distance calculation means preferably calculates the distance from the visible light source to the body surface of the subject based on the distance from the distance detector to the body surface of the subject.

### Action and Effect

According to the X-ray imaging apparatus associated with the present invention, the distance detector comprises the distance detector and the distance calculation means. The distance detector detects the distance from own position at which the distance detector is mounted to the collimator to the body surface of the subject. And, the distance calculation means calculates the distance from the visible light source to the body surface of the subject based on the distance from the distance detector to the body surface of the subject, which is detected the distance detector.

The visible light source control means that controls the light intensity of the visible light which is emitted from the visible light source based on the calculated distance from the visible light source to the body surface of the subject by the distance calculation means. Therefore, the brightness and the region of the exposure field of the visible light irradiated to the body surface of the subject can be suitably corresponding to the distance from the visible light source to the body surface of the subject. Accordingly, the operator does not have to measure manually the distance from the visible light source to the body surface of the subject so that the operator can easily shift the visible light source to the more suitable position for an X-ray imaging while visually recognizing the more appropriate exposure field of the visible light. Consequently, the further suitable X-ray image for diagnosis can be further easily and efficiently obtained.

### Effect of the Invention

The X-ray imaging apparatus according to the present invention comprises the distance measurement means and the visible light source control means. The measurement means measures the distance from the visible light source to the body surface of the subject. Specifically, the operator does not need to measure manually the distance from the visible light source to the body surface of the subject. Consequently, labor and burden needed to the X-ray imaging decrease, so that an efficient diagnosis can be performed.

And, the visible light source control means that controls blinking of the visible light source based on the difference between the distance, from the visible light source to the body surface of the subject, measured by the distance measurement means and a predetermined target value thereof. Accordingly, the operator visually recognizes the exposure field of the visual light, so that the operator can confirm that the visible light source has shifted to the suitable position and the visually recognized exposure field is the optimal exposure field for the X-ray imaging. Specifically, the operator does not have to shift the line of sight to confirm the optimal position of the exposure field for the X-ray imaging. Accordingly, labor and burden to the operator to confirm the optimal position of the exposure field for the X-ray imaging decrease so that the suitable X-ray image for diagnosis can be easily and efficiently obtained.

The above and other aspects, features and advantages of the present invention will become apparent from the following description read in conjunction with the accompanying drawings, in which like reference numerals designate the same elements.

### BRIEF DESCRIPTION OF THE DRAWINGS

FIG. 1 is a schematic view illustrating a structure of an X-ray imaging apparatus according to the aspect of the Embodiment 1.

FIGS. 2A, 2B are schematic views illustrating a structure of a collimator according to the aspect of the Embodiment 1.

FIG. 2A is a cross-section view illustrating a structure of the collimator.

FIG. 2B is a schematic view illustrating a structure of the shielding boards limiting broadening of the X-rays.

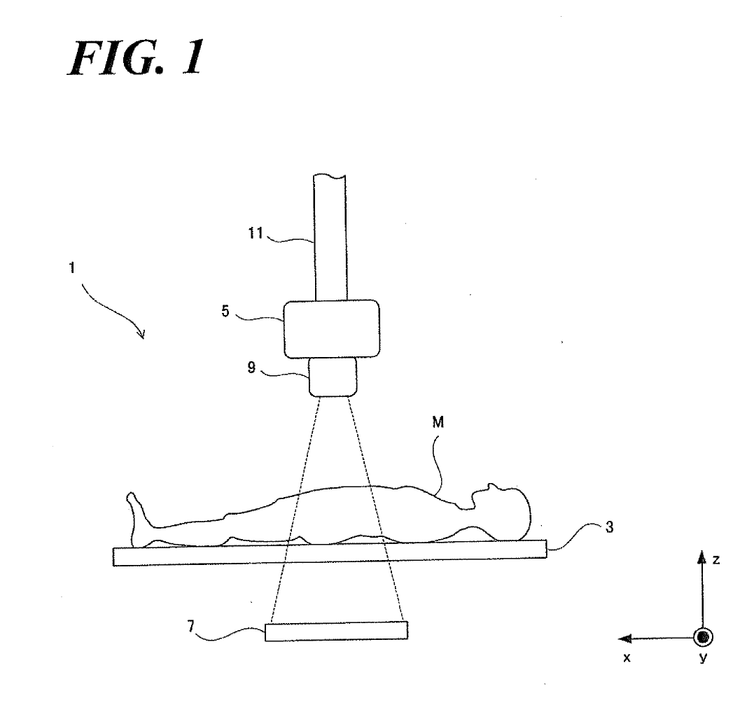

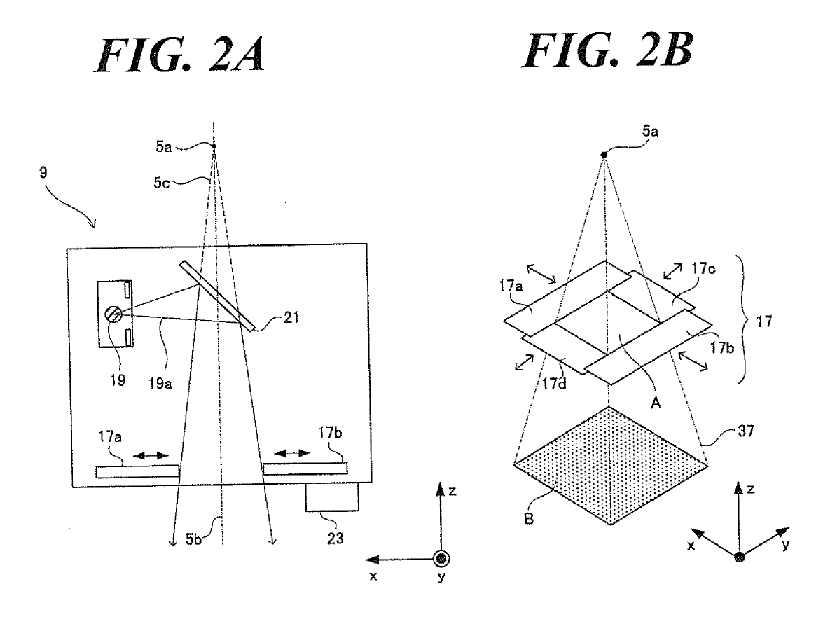

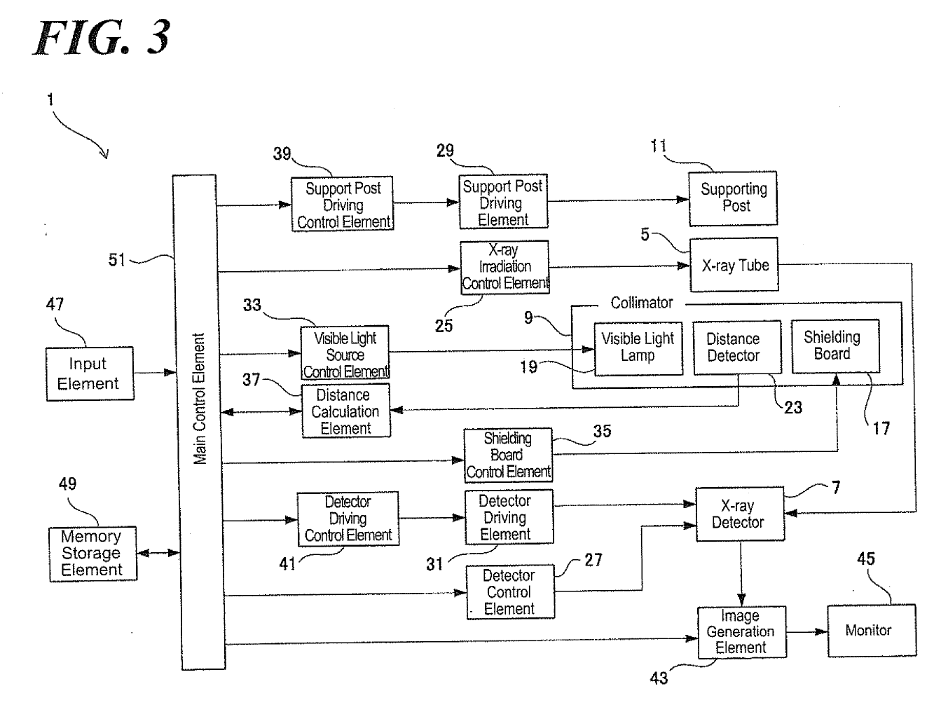

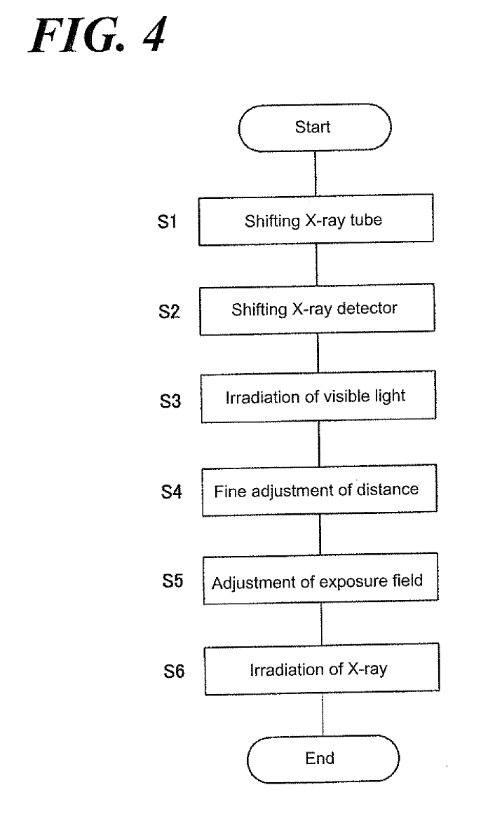

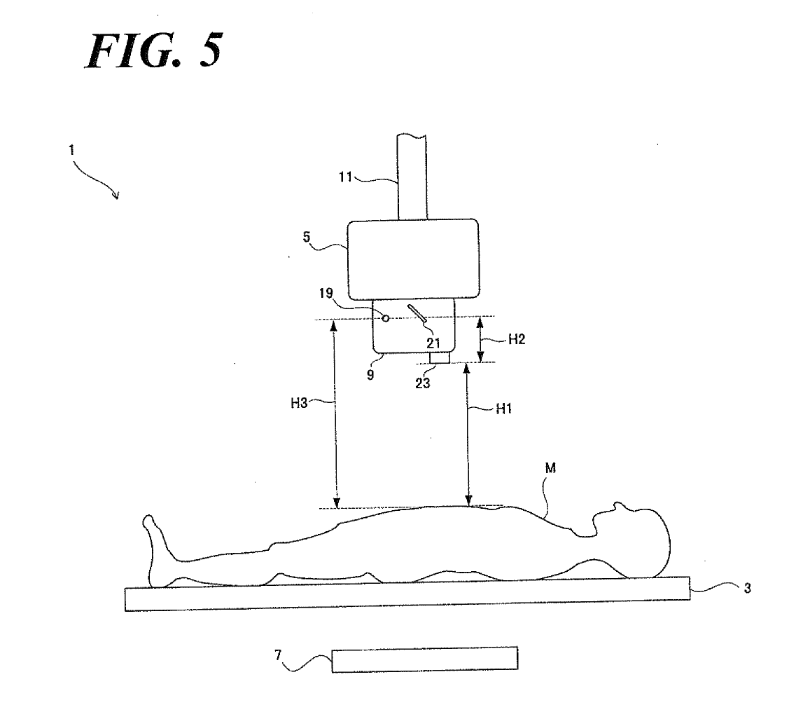

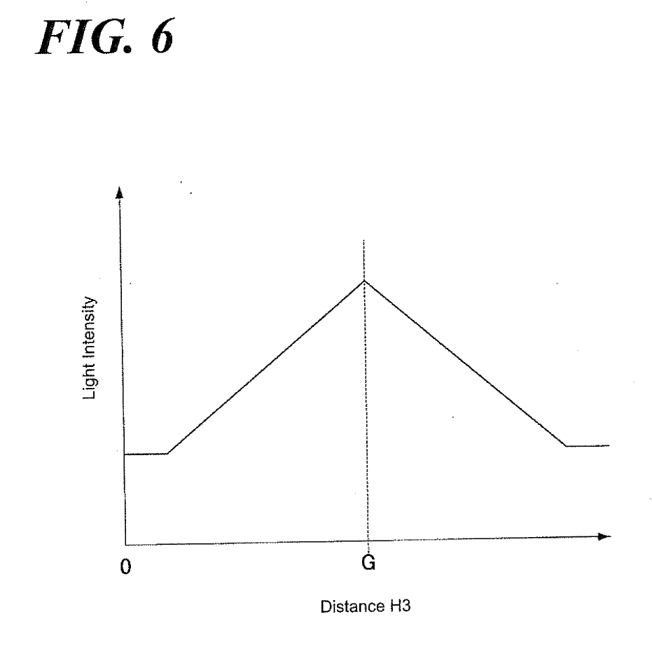

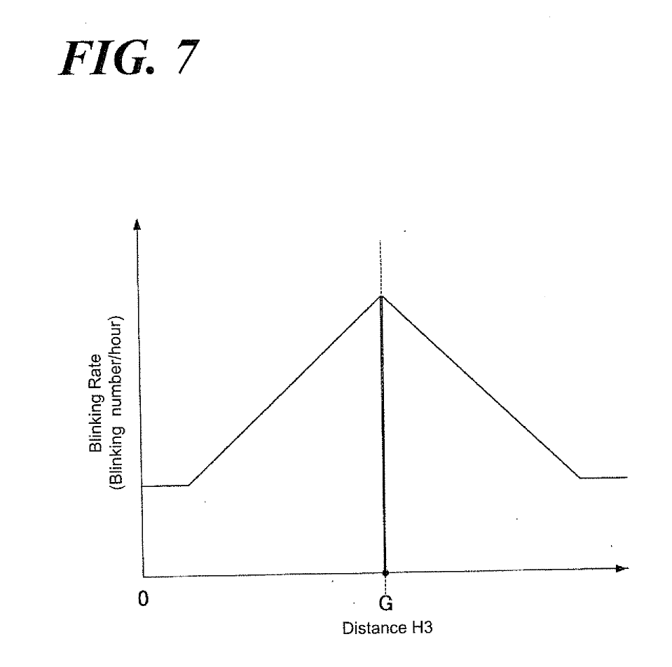

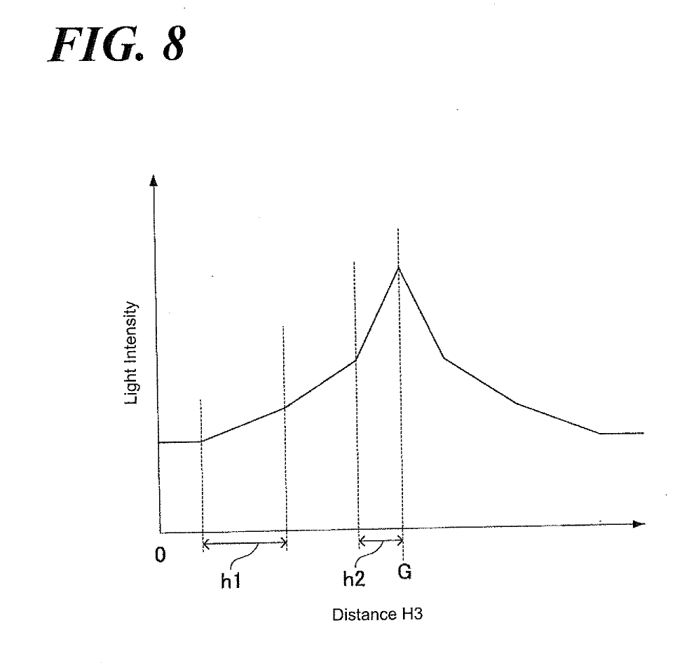

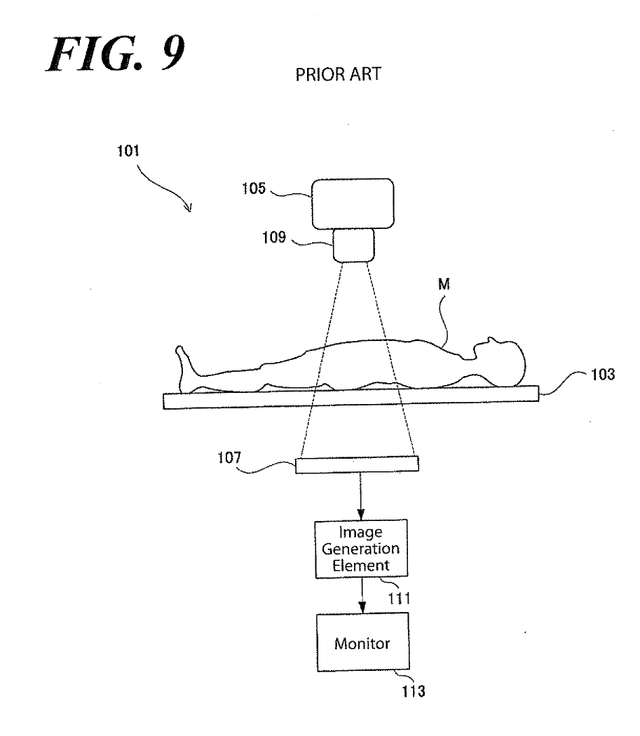

FIG. 3 is a function block view illustrating a structure of an X-ray imaging apparatus according to the aspect of the Embodiment 1.

FIG. 4 is a flow-chart illustrating an operation of an X-ray imaging apparatus according to the aspect of the Embodiment 1.

FIG. 5 is a schematic view illustrating the distance that the distance calculation means calculates relative to the X-ray imaging apparatus according to the aspect of the Embodiment 1.

FIG. 6 is a graph representation illustrating a co-relationship between an intensity of light and the distance calculated by the distance calculation means relative to the X-ray imaging apparatus according to the aspect of the Embodiment 1.

FIG. 7 is a graph representation illustrating a co-relationship between a blinking rate of the visible light and the distance calculated by the distance calculation means relative to the X-ray imaging apparatus according to the aspect of the Embodiment 2.

FIG. 8 is a graph representation illustrating a co-relationship between a light intensity and the distance calculated by the distance calculation means relative to the X-ray imaging apparatus according to the aspect of the alternative Embodiment.

FIG. 9 is a schematic view illustrating a constitution of an X-ray imaging apparatus according to the aspect of the conventional example.

### DETAILED DESCRIPTION OF THE PREFERRED EMBODIMENTS

Reference will now be made in detail to embodiments of the invention. Wherever possible, same or similar reference numerals are used in the drawings and the description to refer to the same or like parts or steps. The drawings are in simplified form and are not to precise scale. The word ‘couple’ and similar terms do not necessarily denote direct and immediate connections, but also include connections through intermediate elements or devices. For purposes of convenience and clarity only, directional (up/down, etc.) or motional (forward/back, etc.) terms may be used with respect to the drawings. These and similar directional terms should not be construed to limit the scope in any manner. It will also be understood that other embodiments may be utilized without departing from the scope of the present invention, and that the detailed description is not to be taken in a limiting sense, and that elements may be differently positioned, or otherwise noted as in the appended claims without requirements of the written description being required thereto.

Various operations may be described as multiple discrete operations in turn, in a manner that may be helpful in understanding embodiments of the present invention; however, the order of description should not be construed to imply that these operations are order dependent.

### Embodiment 1

Referring to the FIGs., the inventor sets forth the Embodiment 1 of the present invention.

Illustration of the entire structure.

Referring to FIG. 1, an X-ray imaging apparatus 1 comprises a table 3, an X-ray tube 5, an X-ray detector 7, a collimator 9, and an image generation element 11. The subject M lies on the table 3 in a horizontal posture. The X-ray tube 5 irradiates the X-ray to the subject M. The X-ray tube 5 and the X-ray detector 7 are in-place facing each other sandwiching the table 3. The X-ray detector 7 detects the X-ray, irradiated to the subject M from the X-ray tube 5, that transmits through the subject M and converts to an electric signal therefrom and then outputs as a detection signal. A flat panel display (FPD) is used as the X-ray detector 7 according to the aspect of the Embodiment 1.

The collimator 9 is installed below the X-ray tube 5. The support post 11 having the base thereof on the ceiling of the examination room and hang-and-support the X-ray 5. Further, the X-ray tube corresponds to a radiation source of the present invention and the FPD 7 corresponds to the detection means of the present invention.

Referring to FIG. 2A, 2B, the inventor further sets forth the structure of the collimator 9. Referring to FIG. 2A, the collimator 9 comprises shielding boards 17, a visible light lamp 19, a perfect mirror 21 and a distance detector 23. The shielding boards comprises; a shielding board 17a, a shielding board 17b that shift in the x-direction (direction of the length of the table 3), and a shielding board 17c and a shielding board 17d that shift in the y-direction (direction of the width of the table 3.) The shielding board 17a and the shielding board 17b shift mirror-image symmetrically in the x-direction as the center axis 5b of the X-ray 5c irradiated from the X-ray focal point 5a is the baseline therefor. And, the shielding board 17c and the shielding board 17d shift mirror-image symmetrically in the y-direction as the central axis 5b of the X-ray is the reference therefor.

Each shielding board 17a-17d are made of a material that can shield X-rays and the material include e.g., lead but not limited thereto. Referring to FIG. 2B, broadening of X-rays irradiated from the X-ray focal point 5a of the X-ray tube 5 is limited by respective shielding boards 17a-17d. Then, the X-rays that pass through the opening A formed with respective shielding boards 17a-17d are irradiated to the subject M. Specifically, the X-ray exposure field B can be adjusted by adjusting the opening A.

Referring to FIG. 2A, a visible light lamp 19 emits the visible light 19a. According to the aspect of the Embodiment 1, a halogen lamp as the visible light lamp 19 is applied but others, e.g., LED lamp, also can be applied thereto. The perfect mirror 21 is configured to irradiate the visible light emitted from the visible light lamp 19 to the subject M. Meantime, the exposure field of the visible light 19a emitted from the visible light lamp 19 is adjusted so as to coincide with the exposure field of the X-rays 5c irradiated from the X-ray focal point 5a by respective shielding boards 17a-17d and the perfect mirror 21. Specifically, the visible light source 19a and the X-ray focal point 5a are geometrically in the conjugate relation. The distance detector 23 detects the distance from the distant detector 23 to the body surface of the subject M. For example, an ultrasound distance meter or a laser distance meter can be used as the distance detector 23. According to the Embodiment, the ultrasound distance meter is used as the distance detector 23. Meantime, the visible light lamp 19 corresponds to the visible light source of the present invention.

Referring to FIG. 3, the X-ray imaging apparatus 1 further comprises an X-ray irradiation control element 25, a detector control element 27, a support post driving element 29, a detector driving element 31, a visible light source control element 33, a shielding board control element 35 and a distance calculation element 37. The X-ray irradiation control element 25 is connected to the X-ray tube 5 and controls the tube voltage and the tube electric current of the X-ray tube 5. According to such controls, the X-ray irradiation control element 25 can control an amount of X-rays irradiated from the X-ray tube 5 and a timing of the X-ray irradiation and so forth. The detector control element 27 is connected to the X-ray detector 7 and controls an operation to read out the electric signal converted by the X-ray detector 7, i.e., X-ray detection signal.

The support post driving element 29 is connected to the support post 11 and shifts the support post 11 in the x-direction and y-direction and expand-and-contracts therefor in the z-direction. When the support post 11 shifts in either x-direction or y-direction, the X-ray tube 5 shifts following the support post 11, so that the imaging position of the subject M can be changed by shifting the support post 11 in the x-direction or y-direction. Further, when the support post 11 contracts in the z-direction, the position of the X-ray tube 5 in the z-direction can be changed. Specifically, the distance between the X-ray tube and the subject M and the distance between the X-ray tube and the X-ray detector can be changed by letting the support post 11 contract. A support post driving element 29 is installed in the upper side of the support post driving element 39, the support post driving element 39 controls the operation of the support post driving element 29.

The detector driving element 31 is connected to the X-ray detector 7 to be shifted in the x-direction, y-direction and the z-direction of the support post 11. The imaging position of the subject M can be changed by shifting the X-ray detector in the x-direction or y-direction. And when the X-ray detector is shifted in the z-direction, the distance between the X-ray tube 5 and the X-ray detector 7 can be changed. The detector driving control element 41 is installed in the upper side of the detector driving element 31, the detector driving control element 41 controls the operation of the detector driving element 31.

The visible light source control element 33 connected to the visible light lamp 19 controls blinking of the visible light lamp 19, diminishing, an amount of light of visible light emitted from the visible light lamp 19 and so forth. The shielding board control element 35 connected to respective shielding boards 17a-17d controls shifting of respective shielding boards 17a-17d. Specifically opening-and-closing of the shielding board 17 is controlled by the shielding control element 35. The distance calculation element 37 is installed in the upper side of the distance detector 23. Then, the distance calculation element 37 calculates the distance from the visible light lamp 19 to the body surface of the subject M in the z-direction (vertical direction) based on the distance between the distance detector 23 and the body surface of the subject M, which is calculated by the distance detector 23.

The X-ray imaging apparatus 1 further comprises an image generation element 43, a monitor 45, an input element 47, a memory storing element 49 and a main control element 51. The image generation element 43 installed in the subsequent part of the detector 7 generates an X-ray image based on the X-ray detection signal output from the X-ray detector 7. The monitor 45 displays the generated X-ray image. The instruction from the operator is input through the input element 47. The input element 47 may include a keyboard, a panel input by touching. Specifically, the visible light source control element 33 corresponds to the visible light source control means of the present invention and the distance calculation element 37 corresponds to the distance calculation means of the present invention. Then, the image generation element 43 corresponds to the image generation means, the distance detector 23 and the distance calculation element 37 corresponds to the distance measurement means of the present invention.

The memory storage element 49 stores a variety of parameters being referred for controlling the X-ray imaging apparatus 1, X-ray images generated by the image generation element 43 and so forth. An example of parameter being referred for controlling the X-ray imaging apparatus may include a parameter of a tube voltage; a tube electric current of X-ray tube 5 controlled by the X-ray irradiation control element 25, a parameter indicating the position of the X-ray tube 5 and the X-ray detector 7 in the z-direction, and so forth. The main control element 51 comprehensively controls the X-ray irradiation control element 25, the detector control element 27, the visible light source control element 33, the shielding board control element 35, the distance calculation element 37, the support post driving control element 39, the detector driving control element 41, and the image generation element 43.

### Detail Description of the Operation

Next, referring to FIGS. 2A, 2B, the inventor sets forth an operation of the X-ray imaging apparatus 1. FIG. 4 is a flow-chart illustrating an operation of an X-ray imaging apparatus according to the aspect of the Embodiment 1.

### Step S1 (Shift of X-Ray Tube)

First, the subject M is laid on the table 3. Then, the operator shifts the support post 11 relative to the subject M by operating the input element 47. Specifically, the data input in the input element 47 are sent to the main control element 51 and the main control element 51 outputs the signal to the support post driving element 39 based on the received sent-data. The support post driving control element 39 controls the support post driving element 29 based on the output signal and shifts the support post 11. The X-ray tube is supported by the support post 11, so that the spacial position thereof relative to the subject M is changed interlockingly with shift of the supporting post 11. The step of the Step 1 is completed by shifting the X-ray tube 5 to the predetermined position.

### Step S2 (Shift of X-Ray Detector)

Next, the operator shifts the X-ray detector 7 relative to the subject M by operating the input element 47. Specifically, the data input in the input element 47 are sent to the main control element 51 and the main control element 51 outputs the signal to the detector driving control element 41 based on the received sent-data. The detector driving control element 41 controls the detector driving element 31 based on the received output-signal and shifts the detector 7. The step of the Step 2 is completed by shifting the X-ray detector 7 to the predetermined position.

### Step S3 (Irradiation of Visible Light)

Following completion of shifts of the X-ray tube 5 and the X-ray detector 7, the operator irradiates the visible light from the visible light lamp 19 to the subject M by operating the input element 47. Specifically, the data input in the input element 47 are sent to the main control element 51 and the main control element 51 outputs the signal to the visible light source control element 33 based on the received sent-data. The visible light source control element 33 controls irradiation of the visible light emitted from the visible light lamp 19 based on the received output-signal. The exposure field of the irradiated visible light is configured to coincide with the exposure field of the X-ray irradiated from the X-ray tube 5, so that the operator can make sure the exposure field of the X-rays by visually recognizing the exposure field of the visible light.

At this time, the operator further operates the input element 47 to turn on the distance detector 23. The turned-on distance detector 23 activates the ultrasound distance meter, structuring the distance detector 23, to transmit ultrasound to the subject M. The ultrasound transmitted from the distance detector 23 is reflected from the body surface of the subject M. Therefore, referring to FIG. 5, the distance detector measures the distance H1 in the z-direction between the distance detector 23 and the body surface of the subject M by receiving the reflected ultrasound. The data relative to the measured distance H1 are sent to the distance calculation element 37.

According to the X-ray imaging apparatus 1 associated with the aspect of the Embodiment, the H2 in the z-direction between the distance detector 23 and the visible light lamp 19 is an eigenvalue. Therefore, the distance calculation element 37 can calculate H3 in the z-direction between the visible light lamp 19 and the body surface of the subject M based on the distance H1 and the distance H2. The data relative to the calculated distance H3 are sent to the main control element 51. The main control element 51 sends the data relative to the distance H3 sent from the distance calculation element 37 to the visible light source control element 33.

### Step S4 (Fine Adjustment of the Distance)

The visible light source control element 33 controls intensity of the visible light emitted from the visible light lamp 19 based on the data received sent-signal relative to the distance H3. As an example, referring to FIG. 6, it is controlled as the smaller the difference between a target value G pre-set as an appropriate value relative to the distance H3 and the H3 actually calculated by the distance calculation element 37 is, the stronger intensity of the visible light emitted from the visible light lamp 19 is. Specifically, when the distance H3 coincides with the target value G, the intensity of the visible light emitted from the visible light lamp 19 becomes a maximum.

The operator visually confirms the exposure field of the visible light and executes a fine adjustment of the position of the collimator 9 with the input element 47. While executing the fine adjustment, the turned-on distance detector 23 measures intermittently the distance H1 in the z-direction between the distance detector 23 and the body surface of the subject M and sends intermittently the data relative to the distance H1 to the distance calculation element 37. Specifically, the distance calculation element 37 calculates intermittently the distance H3 corresponding to the position of the collimator 9, which is being changed, based on the distance H1 and the distance H2.

The operator visually confirms the exposure field of the visible light and executes the fine adjustment of the position of the collimator 9. The operator can confirm that the distance H3 is getting closer to the target value G along with the intensity increase of the visible light emitted from the visible light lamp 19. Then, the intensity of the visible light beam becomes the maximum and the exposure field becomes the brightest illumination, so that it can be confirmed that the distance H3 becomes equal to the target value G.

Further, when the position of the collimator in the z-direction is adjusted by operating the input element 47, the support post driving control element 39 is controlled to shift the support post 11 in the z-direction. The collimator 9 is supported by the support post 11 together with the X-ray tube 5, so that the collimator 9 shifts in the z-direction interlockingly with shift of the supporting post 11. Further, the fine adjustment of the collimator 9 can be executed manually by shifting the collimator 9. When the fine adjustment of the collimator 9 is completed, the value of the distance H3 becomes equal to the target value G, so that the step of the Step S4 can be completed.

### Step S5 (Adjustment of the Exposure Field)

Next, the operator controls opening-and-closing of the shielding board 17 installed to the collimator 9 to adjust the region of the exposure field. Specifically, the data input in the input element 47 are sent to the main control element 51 and the main control element 51 outputs the signal to the shielding board control element 35 based on the received sent-data. The shielding board control element 35 shifts the shielding board 17a and the shielding board 17b in the x-direction and shifts the shielding board 17c and the shielding board 17d in the y-direction. Broadening of the visible light beam is limited by respective shielding boards 17 so that the visible light beam can be irradiated in a constant exposure field. Therefore, the region of the exposure field can be adjusted by shifting of the shielding boards 17a-17d.

Further, an adjustment of opening-and-closing of the shielding board 17 can be also executed by operating a knob, if the knob is attached to the collimator and so forth. Following confirming that the exposure field being irradiated by the visible light beam is in the predetermined range, the operator operates the input element 47 to complete such irradiation of the visible light from the visible light lamp 19. Upon completion of the irradiation of the visible light, the step of the Step 5 is completed.

### Step S6 (Irradiation of X-Rays)

Following the adjustment of the exposure field, the operator operates the input element 47 to instruct the irradiation of X-rays. Specifically, the data input in the input element 47, such as the tube voltage and the tube electric current, are sent to the main control element 51 and the main control element 51 outputs the signal to the X-ray irradiation control element 25 based on the received sent-data. The X-ray irradiation control element 25 activates the X-ray tube 5 to irradiate the X-ray to the subject M based on the received output-signal. The X-ray being irradiated is incident in the same region, relative to the subject M, as the exposure field of the visible light adjusted in the Step 5 and transmits through the subject M and is detected by the X-ray detector 7.

Then, the X-ray detected by the X-ray detector 7 is converted to the electric signal. The electric signal converted by the X-ray detector 7 is read out by the detector control element 27. The read-out electric signal is output from the X-ray detector 7 to the image generation element 43 as the X-ray detection signal, The image generation element 43 generates an X-ray image based on the X-ray detection signal. And, the generated X-ray image is displayed on the monitor 45 and then the entire step associated with the operation of the X-ray imaging apparatus 1 is completed.

### Effects of a Configuration According to the Embodiment 1

Considering such circumstances, according to the X-ray imaging apparatus associated with the aspect of the Embodiment 1, an X-ray imaging apparatus capable of executing an X-ray imaging efficiently with cutting the burden to the operator who measures the distance from the visible light source to the subject can be provided. Hereinafter, the inventor sets forth effects due to the configuration according to the aspect of the Embodiment.

The X-ray imaging apparatus 1 according to the aspect of the Embodiment 1 comprises a distance detector 23 and the distance calculation element 37. And, the distance H3 between the visible light lamp 19 and the body surface of the subject M is sequentially calculated by the distance detector 23 and the distance calculation element 37. Specifically, the operator does not need to measure manually the distance H3 between the visible light lamp 19 and the body surface of the subject M. Accordingly, the distance between the visible light lamp 19 and the subject M can be measured in a short period of time. Consequently, labor and burden needed to the X-ray imaging decrease, so that an efficient diagnosis can be performed. In addition, according to the X-ray imaging apparatus 1 associated with the aspect of the Embodiment 1, the accuracy of the distance H3 to be measured can be improved compared with the manual measurement of the distance H3. Accordingly, a suitable X-ray imaging can be easily performed.

The visible light source control element 33 installed to the X-ray imaging apparatus 1 associated with the aspect of the Embodiment 1 controls sequentially the visible light irradiated from the visible light lamp 19 to the exposure field based on the distance between the visible light lamp 19 and the body surface of the subject M, which is calculated sequentially by the distance calculation element 37. Specifically, it is controlled as the closer the distance between the visible light lamp 19 and the body surface of the subject M is getting close to the pre-set target value thereof, the stronger the intensity of the visible light is. And, when the distance between the visible light lamp 19 and the body surface of the subject M becomes equal to the target value, it is controlled as the intensity of the visible light irradiated to the exposure field from the visible light lamp 19 becomes a maximum.

Therefore, the operator visually recognizes the exposure field of the visible light while adjusting the position of the collimator 9. Then, the intensity of the visible light becomes the maximum, i.e., the exposure field becomes brightest, so that it can be confirmed that the distance between the visible light lamp 19 and the body surface of the subject M has become equal to the target value. Accordingly, the operator can confirm the optimal exposure field for the X-ray imaging without shifting the line of sight from the exposure field by using the X-ray imaging apparatus according to the aspect of the Embodiment 1. As results, labor and burden to the operator to confirm the optimal position of the exposure field for the X-ray imaging decrease so that the suitable X-ray image for diagnosis can be easily and efficiently obtained.

Further, when the distance between the visible light lamp 19 and the body surface of the subject M is getting far away from the target value, the intensity of visible light becomes weaker. Therefore, the operator can easily confirm that the collimator is getting farther away from the subject M than needed. Specifically, the outer housing temperature rising due to increase of the intensity of the visible light emitted from the visible light lamp 19 can be easily avoided. As results, an accident in which the operator gets burn with touching the high temperature outer housing and deterioration of the visible light lamp 19 and the outer housing due to temperature rising can be appropriately avoided.

### Embodiment 2

Next, referring to FIGs., the inventor sets forth the Embodiment 2 of the present invention. Further, an entire structure of the X-ray imaging apparatus according to the aspect of the Embodiment 2 and a flow-chart illustrating the operation steps is the same as for the X-ray imaging apparatus according to the aspect of the Embodiment 1. Accordingly, the inventor skips to set forth the detail while putting the same sign to respective components, the step of operations, but the inventor sets forth a mechanism characteristic relative to the Embodiment 2, in which the visible light control element 33 controls the visible light emitted from the visible light lamp 19.

### Control of Visible Light According to the Embodiment 2

According to an X-ray imaging apparatus associated with the aspect of the Embodiment 1, the visible light source control element 33 controls blinking of the visible light irradiated from the visible light lamp 19 to the exposure field based on the data relative to the distance between the visible light lamp 19 and the body surface of the subject M, which are sent sequentially from the distance calculation element 37. Specifically, the blinking rate of the visible light irradiated to the exposure field from the visible light lamp 19 is controlled corresponding to the difference between the target value G pre-set as the suitable value of the distance H3 and the distance H3 actually sent from the distance calculation element 37. In addition, according to the Embodiment 2, the blinking rate means the number of blinking of the visible light per an hour.

Therefore, according to the X-ray imaging apparatus associated with the aspect of the Embodiment 2, in the Step 4, the visible light source control element 33 controls the blinking rate of the visible light irradiated from the visible light lamp 19 to the exposure field based on the difference between the target value G and the distance H3 actually calculated by the distance calculation element 37. Specifically, referring to FIG. 7, the visible light source control element 33 controls as the smaller difference between the target value G and the actual distance H3 is, the larger the number of blinking of the visible light lamp 19 per an hour becomes. And, it is controlled as when the distance H3 coincides with the target value G, the visible light lamp 19 irradiates continuously the visible light to the exposure field.

While visually recognizing the exposure field, the operator can confirm that the distance H3 is getting closer to the target value G with increase of the blinking rate of the visible light irradiated from the visible light lamp 19 to the exposure field. And, the operator can confirm that the distance H3 is equal to to the target value G, by continuous illumination from the visible light lamp 19 to the exposure field without blinking. The operator confirms that the distance H 3 coincides with the target value G and ends the step of the step S4, and then conducts the step of the Step S5 and thereafter.

### Effects of a Configuration According to the Embodiment 2

According to the X-ray imaging apparatus associated with the aspect of the Embodiment 2, the visible light source control element 33 controls the blinking rate of the visible light irradiated from the visible light lamp 19 to the exposure field corresponding to the difference between the target value G of the distance H3 and the actual distance H3. The visible light source control element 33 controls as the smaller difference between the target value G and the actual distance H3 is, the higher the blinking rate of the visible light lamp 19 is. And, it is controlled as when the distance H3 coincides with the target value G, the visible light lamp 19 irradiates continuously the visible light to the exposure field without blinking.

According to such configuration, the operator is visually recognizing the exposure field of the visible light adjusting the position of the collimator 9 and can confirm that the distance H3 is getting closer to the target value G with increase of the blinking rate of the visible light irradiated from the visible light lamp 19 to the exposure field. Then, the blinking of the visible light illuminating the exposure field is suspended and it can be confirmed that the distance H3 coincides with the target value G by that the visible light is continuously illuminating the exposure field.

In such case, the state in which the visible light is lighting at high blinking rate and the state in which the visible light is continuously illuminating can be easily distinguished. Therefore, the operator can distinguish the state in which the distance H is close to the target value G from the state in which the distance H coincides with the target value G. Accordingly, labor and burden to the operator to confirm the optimal position of the exposure field for the X-ray imaging decrease so that the suitable X-ray image for diagnosis can be easily and efficiently obtained.

The present invention is not limited to the aspect of the Embodiments set forth above and another alternative Embodiment can be implemented set forth below.

(1) According to each Embodiment set forth above, the support post 11 is configured to have the base thereof on the ceiling of the examination room and hang-and-support the X-ray 5, but such configuration thereof is not limited thereto. Specifically, the support post 11 is configured to have the base thereof on the table 3 or the floor of the examination room to support the X-ray tube 5.

(2) According to each Embodiment set forth above, the step order of the step S1 and the step S2 is not limited therein. Specifically, following shifting the X-ray detector in the step S2, shifting of the X-ray tube in the step S1 can be executed.

(3) According to each Embodiment set forth above, the shielding board 17a and the shielding board 17b constituting the shielding board 17 are structured to shift mirror-image symmetrically at the center axis of the X-ray beam as the baseline, but the structure is not limited thereto. Specifically, the shielding board 17a and the shielding board 17b can be structured to shift independently in the x-direction. Further, the shielding board 17c and the shielding board 17d also can be structured to shift independently in the y-direction.

(4) According to each Embodiment set forth above, it is desired that the visible light emitted from the visible light lamp 19 irradiates the exposure field is controlled in the way as described below. Specifically, in accordance with getting a smaller difference between the distance H and the target value G, the visible light source control element 33 controls as the visible light irradiated from the visible light lamp 19 to the exposure field changes farther rapidly.

Here, referring to FIG. 8, the inventor sets forth an alternative Embodiment of the Embodiment 1. Specifically, when the difference between the distance H3 and the target value G is large, the intensity of the visible light changes relatively slowly corresponding to the difference between the distance H3 and the target value G (region of the sign h1.) Specifically, when the difference between the distance H and the target value G is small, the intensity of the visible light changes more rapidly corresponding to the difference between the distance H and the target value G (region of the sign h2.) As an example of a configuration for such control, at least one threshold value is set as for the difference between the distance H and the target value G. Then, the structure in which the visible light source control element 33 switches the connection to the amplifier having a higher gain every time, at which the difference between the distance H and the target value G becomes smaller than each threshold value, should be considered. In such case, the amplifier having a higher gain is connected, so that the change of the intensity of the visible light becomes more rapid.

According to such structure, the smaller the difference between the distance H3 and the target value G is, the more the intensity of the visible light change sensitively relative to change of the difference between the distance H3 and the target value G. Accordingly, the operator can further easily and accurately execute the fine adjustment from the state in which the distance H3 is close to the target value G to the state in which the distance H3 coincides with the target value G. In addition, according to the alternative Embodiment of the Embodiment 2, in such structure, the smaller the difference between the distance H3 and the target value G, the more the blinking rate of the visible light changes rapidly.

### REFERENCE OF SIGN

1 X-ray imaging apparatus
3 Table
5 X-ray tube (X-ray source)
7 X-ray detector (X-ray detection means)
9 Collimator
11 Supporting post
17 Shielding board
17 Visible light lamp (Visible light source)
23 Distance detector
33 Visual light source control element (Visual light source control means)
37 Distance calculation element (Distance calculation means)
43 Image generation element (image generation means)
45 Monitor
47 Input element

It will be understood by those of skill in the complex x-ray imaging apparatus art that the identified elements for input, memory storage, image generating, distance detection, light source control, and all the others, as well as the noted detectors, laps, monitors or other electronic elements include physical electronic circuits, circuit connectors, related electronic and physical connection elements to achieve the identified elements, purpose, and features identified in this disclosure.

Having described at least one of the preferred embodiments of the present invention with reference to the accompanying drawings, it will be apparent to those skills that the invention is not limited to those precise embodiments, and that various modifications and variations can be made in the presently disclosed system without departing from the scope or spirit of the invention. Thus, it is intended that the present disclosure cover modifications and variations of this disclosure provided they come within the scope of the appended claims and their equivalents.

What is claimed is:

1. An X-ray imaging apparatus, comprising:
an X-ray source that irradiates an X-ray to a subject;
an X-ray detection circuit that detects the X-ray irradiated from the X-ray source and transmitted through the subject;
an image generation circuit that generates an X-ray image based on the detection signal output from the X-ray detection circuit;
a shielding board is mounted to a collimator installed between the X-ray source and the X-ray detection circuit and adjusts an X-ray exposure field irradiated from the X-ray source;
a visible light source is mounted to the collimator and emits a visible light to the subject for recognizing visually the X-ray exposure field;
a measurement circuit measures a distance from the visible light source to the body surface of the subject; and
a visible light source control circuit controls a blinking of the visible light corresponding to a difference between the distance, from the visible light source to the body surface of the subject, measured by said distance measurement circuit and a predetermined target value.

2. The X-ray imaging apparatus, according to claim 1, wherein:
said visible light source control circuit controls a light intensity of the visible light, emitted by said visible light source, corresponding to the difference between the distance, from the visible light source to the body surface of the subject, measured by the distance measurement circuit and said predetermined target value.

3. The X-ray imaging apparatus, according to claim 1, wherein:
said visible light source control circuit controls an aspect of blinking of the visible light, emitted by said visible light source, corresponding to the difference between the distance, from the visible light source to the body surface of the subject, measured by the distance measurement circuit and a predetermined target value.

4. The X-ray imaging apparatus, according to claim 3, wherein:
said visible light source control circuit controls a number of blinks of the visible light corresponding to a difference between the distance, from said visible light source to the body surface of the subject, measured by said distance measurement circuit and a predetermined target value.

5. The X-ray imaging apparatus, according to claim 2, wherein:
said distance measurement circuit comprises a distance detector and a distance calculation circuit;
said distance detector detects the distance from its own position at which the distance detector is mounted to said collimator to the body surface of said subject; and
the distance calculation circuit calculates the distance from said visible light source to the body surface of the subject based on the distance from said distance detector to the body surface of the subject.

6. The X-ray imaging apparatus, according to claim 4, wherein:
said distance measurement circuit comprises a distance detector and a distance calculation circuit;
said distance detector detects the distance from its own position at which the distance detector is mounted to said collimator to the body surface of said subject; and
the distance calculation circuit calculates the distance from said visible light source to the body surface of the subject based on the distance from said distance detector to the body surface of the subject.
